(19) United States
(12) Patent Application Publication
(10) Pub. No.: US 2016/0002696 A1
(43) Pub. Date: Jan. 7, 2016

(54) METHOD TO IDENTIFY BACTERIAL SPECIES BY MEANS OF GAS CHROMATOGRAPHY/MASS SPECTROMETRY IN BIOLOGICAL SAMPLES

(71) Applicant: ALIFAX HOLDING SPA, Polverara (IT)

(72) Inventor: Paolo Galiano, Padova (IT)

(73) Assignee: ALIFAX HOLDING SPA, Polverara (IT)

(21) Appl. No.: 14/769,436

(22) PCT Filed: Feb. 20, 2014
(86) PCT No.: PCT/IB2014/059109

§ 371 Date: Aug. 20, 2015

(30) Foreign Application Priority Data
Feb. 20, 2013 (IT) UD2013A000021

(51) Int. Cl. C12Q 1/04; G01N 33/497

(52) U.S. Cl. CPC C12Q 1/04; G01N 33/497; G01N 2033/4977

(57) ABSTRACT

Method to identify bacterial classes in a biological sample, in particular a urine sample, that provides to carry out an analysis by means of Gas Chromatography-Mass Spectrometry (GC/MS) of the volatile components, such as metabolites and catabolites, of the sample in order to identify a graphic plot characteristic of a specific bacterial class.

### FIELD OF THE INVENTION

The present invention concerns a method to identify bacterial classes in biological samples, in particular, but not only, urine samples, by means of techniques based on gas chromatography/mass spectrometry (GC/MS).

### BACKGROUND OF THE INVENTION

The rapid and accurate microbic identification in a biological sample is fundamental in the diagnosis of infectious diseases, and therefore in the formulation of the correct antibiotic therapy.

Different methods, both direct and indirect, are known in the state of the art for the identification of pathogenic bacteria in biological samples of patients.

The main direct method, currently held the “gold standard”, consists in the identification through biochemical and morphological tests of the bacterial colonies obtained from culture in specific media.

The culture techniques, depending on the medium used (for example in classical culture media), are either completely generic (for example the technique called Cled) or, in a different way, are able to select more or less thoroughly the characteristics of the colonies identified.

Among these, techniques are known that use fermentation of glucose and lactose (see for example the MacConkey culture medium), and the capacity of partial or total haemolysis of the red corpuscles (for example the method that uses blood agar plates). Some groups or bacterial classes can be traced back to these characteristics.

One variable is represented by the presumptive identification techniques by sowing samples on Petri dishes containing chromogenic media able to pigment the various types of bacterial classes in a differential way. In these cases, the differentiation is the visual type based on different colors of the colonies that develop in the culture.

The classic identification of the sample is obtained by analyzing, with manual or automated systems, a suitable pattern of biochemical tests that is tested by inoculating a standardized bacterial suspension (0.5 McFarland concentration) obtained with the colonies previously isolated on Petri dishes (indifferently with the various types of colonies cited above).

Recently, different analytical techniques have been developed instrumental to improve the speed and precision of identifying bacterial cells. In these techniques, the biochemical components of the bacterial cells, such as lipids, phospholipids, lipopolysaccharides, oligosaccharides, proteins or nucleic acids are examined to determine specific taxonomic markers for each bacterium.

Molecular biology techniques, such as amplification, hybridization and sequencing of nucleic acids combine the characteristics of specificity, sensitivity and rapidity of result, and are causing ever greater interest in microbiology laboratories. However, as of today they are still not very standardized methods, and are also extremely expensive.

Flow cytometry has been proposed as a technique for identifying bacteria in biological samples, but experimental evidence has shown poor performance in terms of specificity and positive predictive values (Tuesta, ECCMID 2009). Other luminescence techniques proposed are also limited to the study of pure colonies, or must be associated with immunological methods.

Among the most modern techniques for identifying bacteria on biological matrices, the application has been developed which provides to use Raman spectroscopy, based on molecular vibrations. This is a non-destructive and non-invasive analytical technique for analyzing biological materials, including integral bacteria, due to the high specificity and resolution of the vibrational spectrums and the weak background signal from the water environment typical of living systems. The Raman signal is however relatively weak and is amplified using the SERS technique, which exploits the adsorption of the analytes on special metal resonators (silver, gold and copper) (see for example CA 2668259A1).

In recent years mass spectrometry with its various combinations, for example associated with gas chromatography or pyrolysis methods, seems to be the most promising technique for the development of methods for the rapid identification of bacteria with high sensitivity and specificity. Descriptions of such techniques that use mass spectrometry have been reported in patent documents which provide, for example, to study the proteomic profile (WO 2009/065580 A1), rather than the lipid profile (US 2011/086384, WO 2008/058024A2) or the genetic profile (U.S. Pat. No. 5,605,798) or the metabolomic profile (WO 2011/064000 A1).

In 1996 a method was perfected for identifying bacteria based on a particular technique of mass spectrometry called MALDI-TOF (Matrix-Assisted Laser Desorption Ionization—Time of Flight).

All the mass spectrometry techniques allow to measure the molecular weight of the molecules present in a sample but, unlike other techniques, MALDI-TOF mass spectrometry allows to analyze samples consisting of complex matrices. Using this technique, it has been possible to analyze bacterial cells for the first time. The result of this analysis is a mass spectrum whose signals originate both from protein components weakly linked to the cell wall, and also from molecules released following a partial lysis of the molecule during the analytical stage. The molecular weight of these protein components varies from class to class, and the mass spectrums obtained by analyzing bacterial cells represent an extremely specific fingerprint, closely correlated to the bacterial class analyzed.

The technique provides to take a certain number of colonies from the Petri dish and to position them on the appropriate well of the laser irradiation platelet of the instrument.

Subsequently, a suitable matrix is added and the platelet is made to dry by thermostating at 37° C. before the laser irradiation and registration of the mass spectrum by the instrument.

A variant of the method described above (WO 2009/065580A1 in the name of Brucker), provides an application on a positive hemoculture flask, the material of which is centrifuged and the pellet obtained is positioned on the appropriate well of the sample-carrying platelet of the instrument. The method then continues as described before.

Known patent documents provide to analyze the proteomic, lipid or genetic profile of isolated colonies of bacteria, or in any case of a bacterial pellet obtained from the centrifuged material of biological sample.

Document WO 2011/064000 describes a method to monitor, identify or diagnose an infection caused by bacteria in an animal biological sample (rats) using the comparative analysis of the metabolic profile of the sample with respect to a control sample using a GC/TOF/MS system.

In the animal model analyzed and described, the overall metabolic profile, also called metabolomic, was used to detect the variations in abundance of biomarkers for monitoring the health of the infected subject.

The same document also proposed a method for monitoring, identifying or diagnosing a bacterial infection based on analysis using a GC/TOF/MS system, after extraction with solvent from serum of the components having low molecular weights. The data obtained do not allow, however, to identify specific metabolites of the bacterial strains, but super- or under-expressions of the metabolites present in the serum. Multivarious analyses of the data only underline that these changes can be related to the bacterial strain.

More recently a new analytical approach has been perfected, based on the concentration of the volatile metabolites/catabolites of bacteria grown in a suitable culture broth on a solid phase micro extraction (SPME) support, and subsequently analyzed by GC/MS. This system has given satisfactory results, showing how different bacteria lead to different metabolic/catabolic profiles, thus allowing them to be identified in biological substrates. One disadvantage of this system is essentially connected to the times and costs required for the SPME concentration step.

The article by Heather D. Bean et al, published on 30.05.2012: “Bacterial volatile discovery using sold phase microextraction and comprehensive two-dimensional gas chromatography time-of-flight mass spectrometry” describes the study of the profiles of the bacterial catabolites after extraction from the culture broth using centrifugation; the supernatants are analyzed using SPME after filtration.

The article by Khalid Muzaffar Banday et al, published in 2011: “Use of urine volatile organic compounds to discriminate tuberculosis patients from healthy patients” describes a method that allows to obtain a discrimination between patients affected with tuberculosis and healthy patients. The markers found in the urine are not products of the metabolism/catabolism of bacteria on a urine level, but are dismetabolism products on a systemic level due to the development of tuberculosis.

The article by T. J. Davies et al, published in 1984: “VOLATILE PRODUCTS FROM ACETYLCHOLINE AS MARKERS IN THE RAPID URINE TEST USING HEAD-SPACE GAS-LIQUID CHROMATOGRAPHY”, describes a method in which the urine bacterial metabolites/catabolites as such are not analyzed, but acetylcholine is introduced into the biological sample and the products of the bacterial metabolism of this substance are studied.

In WO 2004/081527, published in 2004, “Systems for differential ion mobility analysis”, methods based on Ion Mobility, not on mass spectrometry, are described.

The need has therefore arisen, and is a purpose of the present invention, to develop a new technique for identifying bacteria in biological samples, in particular urine samples, which exploits the combined technique of head-space (SHS) gas chromatography/mass spectrometry (GC/MS) so as to obtain more significant, precise and rapid results compared with what can be obtained with current techniques.

Another purpose of the invention is to allow to identify bacterial classes using native urine samples.

Another purpose is to obtain the identification of bacterial strains even in infected urine samples without using growth and enrichment medium.

Another purpose is to allow information concerning the co-presence of mixed bacterial colonies present in biological samples without said co-presence influencing the reliability of the identification.

The Applicant has devised, tested and embodied the present invention to overcome the shortcomings of the state of the art and to obtain these and other purposes and advantages.

### Definitions

A terminology will be used in the present description for which a preliminary definition must be given.

Graph of the liquid growth medium: this means the graph of the various peaks of substances detectable by the gas chromatography/mass spectrometry system (hereafter abbreviated to GC/MS) of the culture broth, also called broth plot (BP).

Metabolic peaks: these are the peaks of the GC/MS plot that identify substances present in the culture broth that the bacterial class has consumed as nourishment for its replication (MPP).

Catabolic peaks: these are the peaks of the GC/MS plot that identify substances produced by the bacterial class (CPP).

Metabolomic library: this is the combination of the GC/MS plots comprising various specific peaks of each bacterial class in reference to the substances consumed for its metabolism or growth (ML).

Catabolomic library: this is the combination of the GC/MS plots comprising various specific peaks of each bacterial class in reference to the substances produced by its catabolism (CL).

Selective solid medium: this is a solid growth medium to which selective action substances (SSM) have been added.

Selective liquid medium: this is a liquid growth medium to which selective action substances (SLM) have been added.

Graph of non-infected native urine: this is the combination of the various peaks of substances detectable by the GC/MS system in non-infected native urine, in the absence of any cultural medium (NUP).

Graph of infected native urine: this is the combination of the various peaks of substances detectable by the GC/MS system in infected urine, in the presence or absence of cultural medium (CNUP).

Static headspace: this is the volume of the container (vial) above the bacterial culture (SHS).

### SUMMARY OF THE INVENTION

The present invention is set forth and characterized in the independent claims, while the dependent claims describe other characteristics of the invention or variants to the main inventive idea.

The present invention provides a method to detect and identify bacterial classes in biological samples using an SHS/GC/MS detection and analysis system of the volatile substances generated by the bacteria following their catabolism and/or metabolism.

In a preferential form of embodiment, the biological samples, for example but not only, native urine or native urine to which liquid culture media have been added, are inserted in a suitably sealed vial; the volatile substances which are then to be subjected to GC/MS analysis are sampled, using a suitable pick-up system, in the volume above (headspace) the biological sample.

In a preferential form of embodiment, a fixed quantity in volume of the volatile sample is then sent to the gas chromatographic injector for GC/MS analysis.

In another preferential form of embodiment, to pick up the volatile substances a needle system is used that perforates the closing element of the vial and takes in a desired quantity of gaseous mass above the biological sample inside which the volatile substances generated by the bacteria are present.

The present invention is based on the principle that live bacteria have their own specific metabolism and catabolism of the substances used as the source of growth (metabolites) and substances produced (catabolites).

According to the invention, at least one step is then provided in which the volatile metabolites and catabolites are picked up in a volume above a bacterial culture, disposed inside a vial in which the biological sample, in particular urine, to be analyzed, has been introduced, inoculated or sown.

According to a variant, the volatile metabolites and the catabolites are picked up using the technique of solid phase microextraction (SPME).

The invention then provides at least a step in which the volatile metabolites/catabolites are analyzed using GC/MS.

The purpose of this analysis is to identify the presence of metabolic markers and catabolic markers for each bacterial class in order to construct a specific metabolomic profile (MPP) and a specific catabolomic profile (CPP). The metabolomic and catabolomic profiles, through comparison with a plot relating to the culture medium or broth alone (BP), allow to constitute specific libraries relating to the various bacterial strains, which then allow to obtain identification in the analysis of a biological sample in the search for possible bacterial infections present therein.

Using this method, the invention allows to identify, in a relatively short measuring time (in the range for example of 5 minutes per sample), the bacterial classes present in the biological sample analyzed, and therefore allows to start a possible targeted antibiotic therapy.

The invention can also be easily integrated in other automatic systems in order to provide results suitable for automation in microbiology.

In the case proposed here, but not restrictively, it has the following operating advantages over other laboratory techniques.

For bacterial identification, the known MALDI-TOF system requires a pellet to be prepared from bacterial isolated material. The limitations of the MALDI-TOF method, as known to all persons of skill, are given by detection limits when mixed colonies appear, that is, more than one colony present in the pellet.

The method according to the present invention does not require any pellet to be prepared, since the examination of the headspace is performed directly in the sealed vial where the sample to be examined has been inoculated. The analysis of the volatile substances taken thus supplies a specific chromatographic plot for each bacterial class and is less influenced by the presence of more than one bacteria present in the same sample.

In fact, since every bacterial class expresses its metabolomic/catabolomic components irrespective of the co-presence of other strains, the present invention allows to obtain information also on the presence of mixed colonies, something which is not possible, or is extremely difficult, to obtain with the known techniques mentioned above.

The present invention, as we said, does not require any specific preparation of pellets, that is, it does not need any handling of the sample and corresponding centrifugation in order to obtain said pellets.

The present invention allows to supply a totally automatic system for bacterial identification, with the possibility of a subsequent specific antibiogram for every single bacterial class identified.

The analysis of the headspace of the sample to be analyzed can be supplied by a native sample, or a native sample to which liquid growth medium has been added, or by an isolated colony from a Petri dish diluted in liquid medium.

### 1) Procedure and Preparation of Urine Samples in Liquid Medium

Known strains of ATCC (American Type Culture Collection) microorganisms were added to native urine samples from healthy patients and inserted in vials containing liquid medium broth. The sample was then detected at various levels of McFarland turbidity and subsequently analyzed using the GC/MS technique.

The result of the analysis showed a catabolomic profile (CPP) characteristic of the ATCC strain added.

The result also showed a metabolomic profile (MPP) characteristic of the ATCC strain added as a difference with respect to the BP graph.

### 2) Procedure and Preparation of Urine Samples in Petri Dishes

Known strains of ATCC microorganisms were added to native urine samples from healthy patients. The sample thus obtained was sown on Petri dishes. Then the sample was taken from the culture medium with a calibrated loop for isolated colonies and diluted on the liquid medium. Subsequently, the sample was detected at various levels of McFarland turbidity and subsequently read using the SHS/GC/MS technique.

In this case too, the result of the analysis showed both a catabolomic profile (CPP) characteristic of the ATCC strain added, and a metabolomic profile (MPP) characteristic of the ATCC strain added, as a the difference with respect to the BP graph.

### 3) Procedure and Preparation of Native Urine Samples

Known strains of ATCC microorganisms were added to native urine samples from healthy patients. The sample was then detected at various levels of McFarland turbidity and subsequently read using the GC/MS technique. The result of the analysis showed a catabolomic profile (CPP) characteristic of the ATCC strain added.

### Description of the Results

Using this technique, it is clear that the invention allows to detect catabolic peaks (CPP) and metabolic peaks (MPP) specific for urine samples containing ATCC bacterial strains sown in solid medium or Petri dish or liquid growth medium, or for native urine samples.

The invention also allows to identify bacterial classes present in urine samples using samples without any pre-treatment whatsoever before reading in the GC/MS instrument, that is, without needing to centrifuge the sample to obtain a concentrated pellet of bacteria.

The invention therefore allows bacterial identification by means of which responses can be obtained in automatic flow systems.

The invention allows to identify metabolomic peaks (MPP) and catabolomic peaks (CPP) for each individual bacterial class, providing peculiar GC/MS plots able to allow the construction of specific metabolic libraries (LB) and catabolic libraries (CL) for each bacterial class.

The metabolic peak (MPP) supplies the plot of the substances consumed by the bacterial strains through comparison with the plot “Liquid medium graph (BP)” as bacterial nourishment.

The invention therefore allows to use specific libraries for each bacterial class both using solid growth medium (Petri dishes) and also using liquid medium and also from native samples without culture medium.

The bacterial strains present in the urine show libraries specific for metabolites (ML) and also libraries specific for catabolites (LB).

The detection of the metabolic peaks (MPP) of the urine samples and the catabolic peaks (CPP) of the urine samples was performed at different levels of McFarland turbidity, in order to identify the optimum values of bacterial titer for the subsequent GC/MS analysis.

The invention, as we said, also allows to obtain bacterial identification in native urine samples without any growth medium.

Analytical procedures: as explained above, the method according to the present invention is based on the analysis of volatile metabolites/catabolites present in the volume above, that is, the headspace, of a sealed container, for example a vial, where the bacterial culture is contained. The volatile molecular classes are taken using the SHS system or, according to a variant, using the SPME system, which allow a direct analysis by means of GC/MS, without any treatment of the sample.

To obtain a drastic reduction in the analysis times, it was therefore provided to perform an accurate parameterization of the SHS system and to use fast chromatography (Fast GC). The typical times of the chromatographic analysis of 30-35 minutes for the SPME/GC/MS system were reduced to 7-10 min.

In a first step the samples relating to the culture broth were analyzed, so as to obtain a blank. Then, samples of medium+bacterial strains were analyzed at different McFarland values.

The analysis of the GC/MS plots specific for every bacterial strain allowed to identify characteristic molecular classes of every bacterium (catabolites) and to show considerable reductions in the abundance of classes characteristic of the culture broth (metabolites).

In a preferential solution, the detection of characteristic classes for every bacterial strain, characterized by a precise chromatographic retention time and mass/load ratio (m/z), was performed using the technique known as “reconstructed ion chromatogram” (RIC).

### ILLUSTRATION OF THE DRAWINGS

The attached drawings are given as a non-restrictive example, and show some diagrams:

FIG. 1 shows the GC/MS plot of the liquid growth medium compared with a GC/MS plot of broth containing a bacterial strain Escherichia coli;

FIG. 2 shows a series of RIC plots showing the reduction of the nutritional or metabolomic peaks as an effect of their metabolic nutrition;

FIGS. 3-6 show examples of plots of the catabolic peaks of specific substances present in ATCC bacterial strains as the product of their catabolism;

FIGS. 7-9 show the RIC plots relating to the classes with m/z 108 (characteristic for K. pneumoniae), with m/z 88 (characteristic for E. faecalis) and with m/z 162 (characteristic for E. coli);

FIGS. 10-14 show plots obtained for native urine, infected respectively with the bacterial strains of E. coli, E. faecalis, K. pneumoniae, P. mirabilis and S. epidermidis.

### DESCRIPTION OF THE DRAWINGS

Hereafter we shall describe, by way of example, some examples of the analysis of bacterial strains cultivated (inoculated) in a growth medium, and will show in the drawings the analytical results obtained.

Known bacterial strains from ATCC strains were reconstituted and incubated in a liquid culture medium containing a mixture of peptones able to make the bacteria replicate.

The bacterial growth was monitored by measuring the level of McFarland turbidity in order to know the level of growth.

Measuring the turbidity allowed to classify various suitable McFarland levels.

Once the development of the bacterial growth had been detected in the liquid culture broth, quantities of cultural broth containing the bacterial strains were taken for each bacterial class examined.

The volatile components present in the headspace were taken by SHS and automatically injected into the GC/MS instrument.

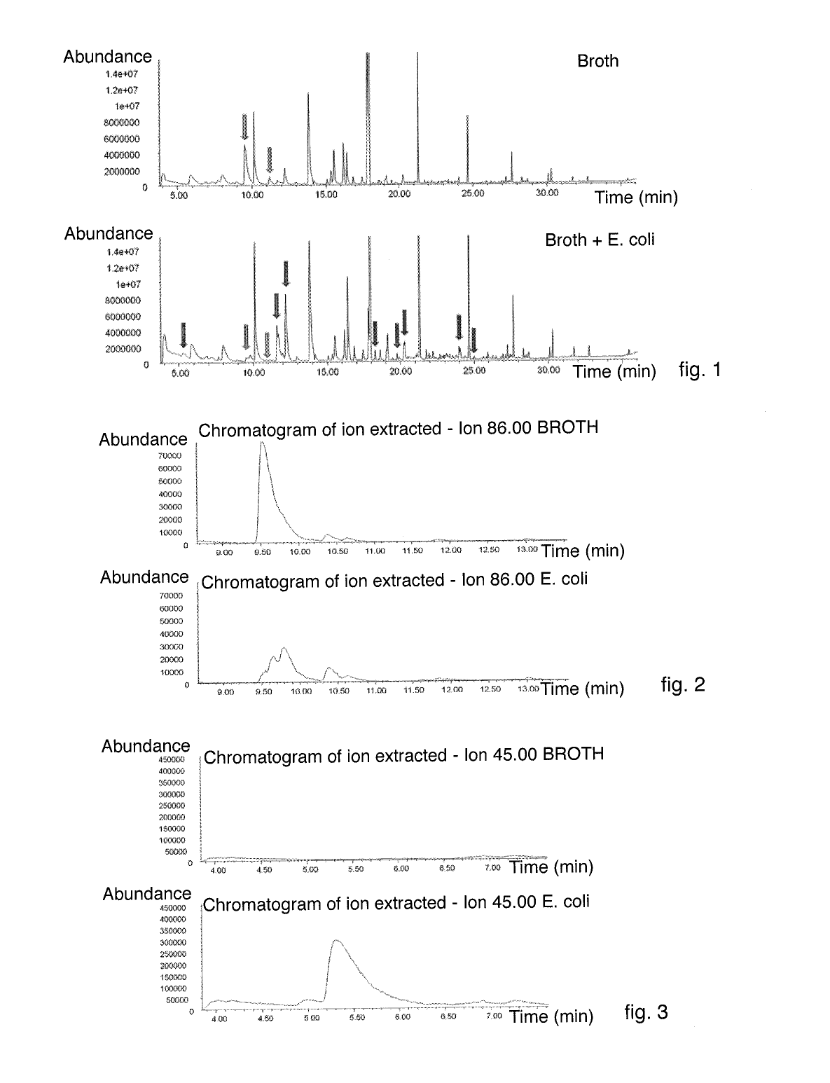

FIG. 1, in the upper part, shows the GC/MS plot obtained from the liquid growth medium. The volatile substances emitted by said liquid growth medium were analyzed using GC/MS and the resultant plot revealed various peaks of specific substances. It is defined as the “Growth medium graph” or “Broth plot” (BP) or “Blank plot”. In the lower part it shows the plot obtained from the substances taken in the headspace of the same broth in the presence of Escherichia coli (1 McFarland).

### Graph of Metabolic Peaks

Known ATCC bacterial strains were inserted (sown) in the liquid growth medium and at various McFarland values the volatile substances present in the headspace were injected into the GC/MS instrument.

The bacteria present expressed chromatographic plots with specific peaks of metabolized substances, that is, substances subtracted from the liquid growth medium as source of nourishment.

The nutritive or metabolomic peaks expressed in said plot underline their specificity of substances subtracted from the broth, inasmuch as from the “Broth plot” (BP) the peaks decreased as an effect of their metabolic nutrition. On this point reference should be made to the comparative plots shown in FIG. 2 where, here too, the upper part of the graph shows the plot relating to the culture medium alone, whereas the lower part shows the plot in the presence of sowing in the medium of the strain of Escherichia coli.

The peaks can be defined as “bacterial metabolomic graph” and, compared with the graph relating to the culture broth alone, or “broth plot”, showed the consumption, or rather the reduction, of various peaks detectable only in the culture broth, as is shown for example in the interval of time between 9.50 and 10.00 minutes.

In other words, measuring with GC/MS allowed to show, by means of specific peaks, the metabolism of said bacteria and, by comparison with the peaks of the culture broth alone, it also allowed to show the detection of the substances that the bacteria feed on by relative reduction of the specific peaks with respect to the broth alone.

Having shown that the analysis of the nutritional bacterial metabolites is characteristic for each bacterial class, the detection of specific peaks allows to write a “Metabolomic library” relating to each bacterial class.

### Graph of Catabolic Peaks

The same analysis and comparison were carried out with ATCC bacterial strains, which gave catabolic peaks of specific substances as the product of their catabolism, as can be seen from the plots shown in FIGS. 3-6.

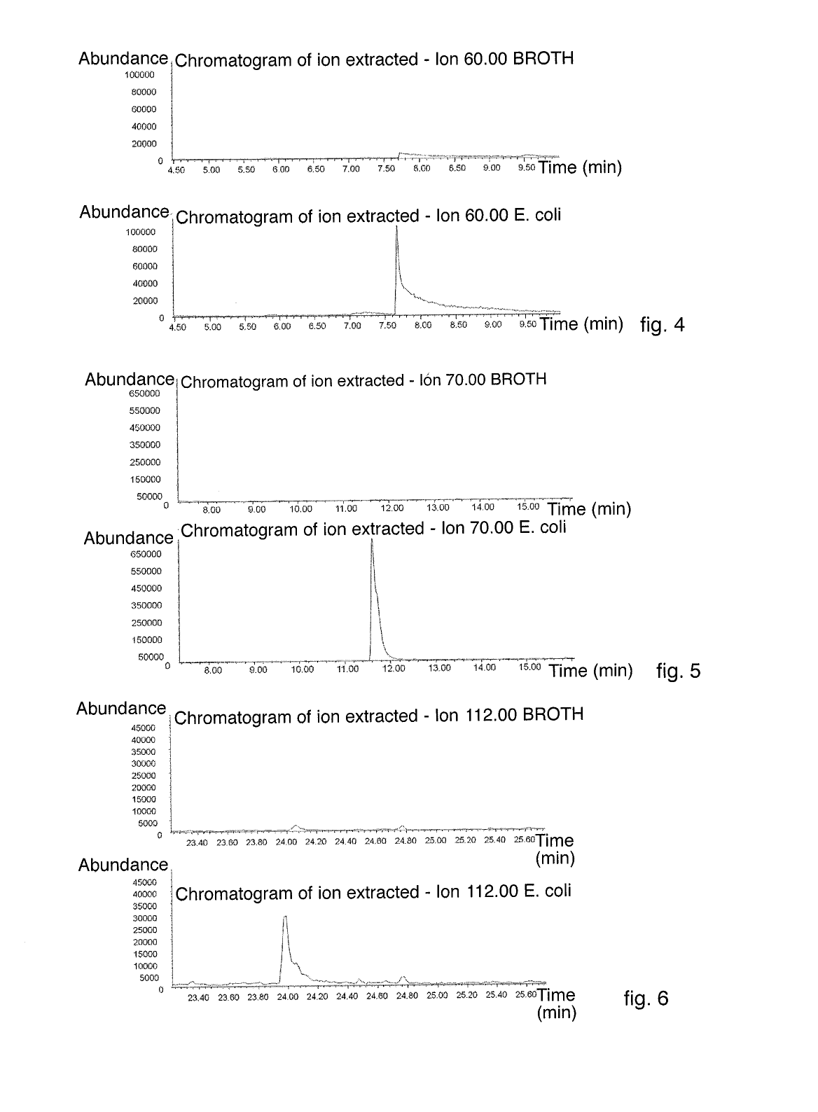

As before, the drawings show the development of the GC/MS plots obtained with broth alone (upper part) and with the presence of bacterial strains (Escherichia coli) with various values of the ratio mass/bacterial load (m/z) (FIG. 3: m/z=45; FIG. 4: m/z=60; FIG. 5: m/z=70; FIG. 6: m/z=112).

In this case too, the analysis of the bacterial catabolites, which is characteristic for each bacterial class due to the presence of specific peaks, allowed to write a “Catabolomic Library” of each bacterial class.

In conclusion, by using the GC/MS instrument, it was possible to obtain, for each bacterial class, a “Metabolomic Library” and a “Catabolomic Library”, which could then be used as an instrument of comparison in identifying the bacterial classes to be sought.

The invention in any case also allows to use the two libraries separately; for example, the “Catabolomic Library” alone may be sufficient to identify the bacterial class, since it may give an unequivocal recognition of a bacterial strain and can be used by taking the sample directly from the Petri dish or from the native urine.

To validate this technique, for example 30 different bacterial strains were then analyzed.

Each bacterial class analyzed with GC/MS provided peculiar graphs, characteristic of plots relating to metabolic peaks and catabolic peaks.

The plots, as we said, allowed to create specific libraries for each bacterial class.

Having identified the peaks characteristic of each bacterial class, the analysis time was reduced to the time necessary for obtaining the final plot, able to detect characteristic peaks, in this way allowing to obtain analysis times compatible with the daily routine.

From the above it is clear how the invention allows to analyze and identify different bacterial classes by means of the dynamic of the bacterial metabolism of each class and of its catabolism, for samples of bacterial strains that have, in GC/MS measuring dynamics, specific graphs of peaks referring to the catabolites of every individual class and, at the same time, graphs of peaks referring to substances subtracted from the culture broth.

It was found that the graphic plot relating to each bacterial class has peculiar peaks both for the metabolites produced and also for the substances consumed by the culture broth in which the bacterium was inoculated in order to facilitate growth.

The plot of each bacterial class, both for the detection of the metabolites consumed and for the detection of the catabolites produced, is specific for each bacterial class and therefore allowed identification by analyzing the relative graphs attributable to data, that is, by comparison with reference data contained in a library of each bacterial class.

To obtain a dynamic analysis of the individual bacterial classes using the “Catabolomic Library”, the preferential but not restrictive solution is to use a liquid growth medium.

The invention therefore expresses, in its dynamics during the GC/MS measurement, the comparison and detection using a chromatogram of the substances that the bacterial class fed on, that is, bacterial metabolism, and its catabolism, or the substances detected by its specific catabolism.

The same approach was used to analyze broth containing urine infected by 30 different bacterial strains. Metabolomic and catabolomic profiles were observed, to some degree different from those observed in the presence of the culture broth alone, but in this case too it was possible to identify molecular classes originating from the bacterial catabolism in the presence of urine.

Some of these classes are specific for each bacterial strain, thus allowing to produce a library containing the data relating to bacterial growth in the broth plus urine system. The library can be used to identify specific bacterial classes present in infected urine samples.

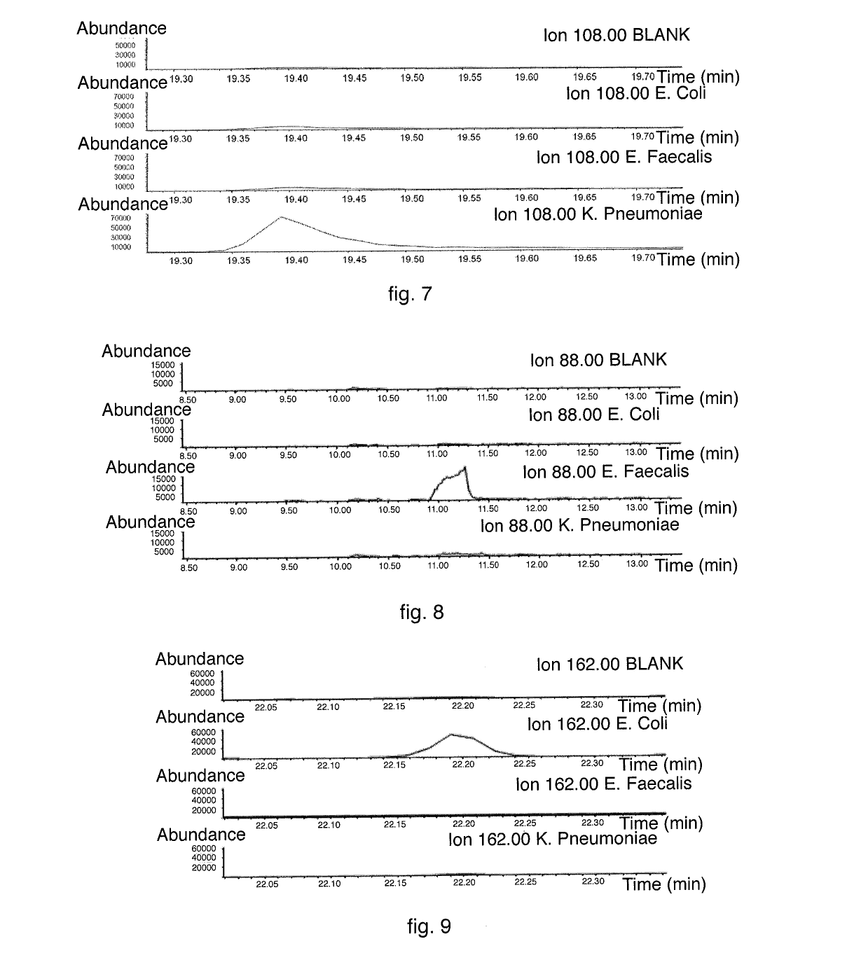

For example, FIGS. 7-9 show the RIC plots relating to the classes with m/z 108 (characteristic for k. pneumoniae), with m/z 88 (characteristic for E. faecalis) and with m/z 162 (characteristic for E. coli), compared with those obtained from samples of broth plus non-infected urine.

The validity of the method described above was also tested on native urine samples, in the absence of any culture medium.

The results obtained show that in these conditions too the volatile catabolites, specific for each bacterial strain, can be detected.

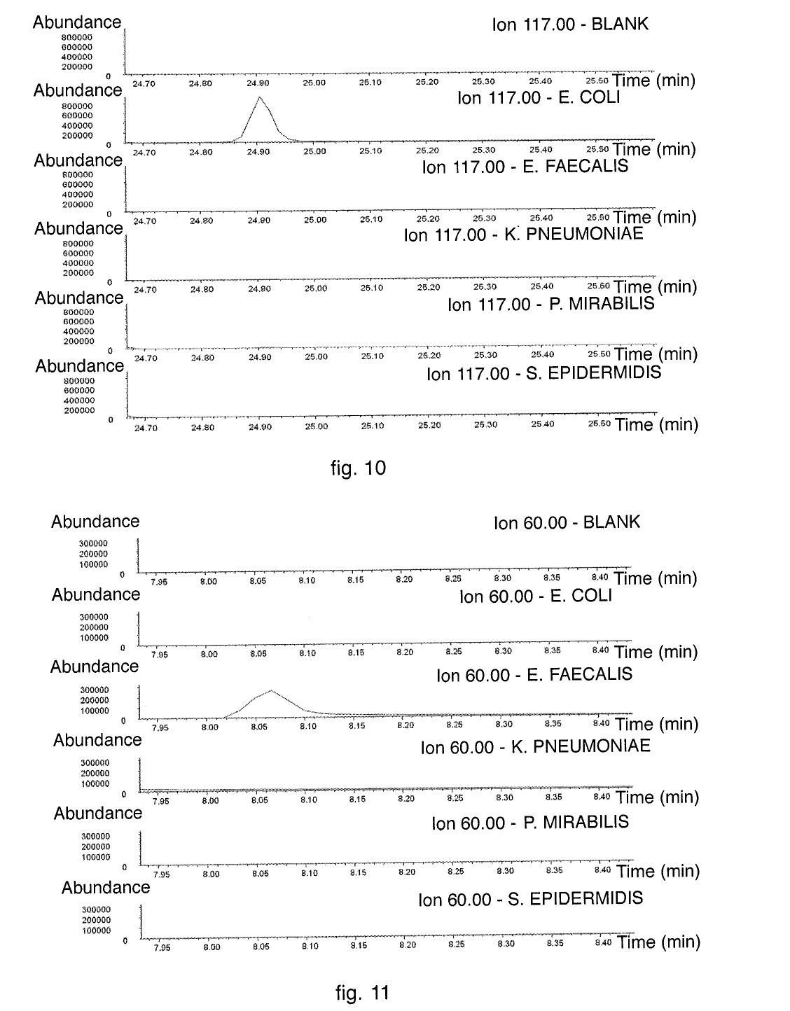

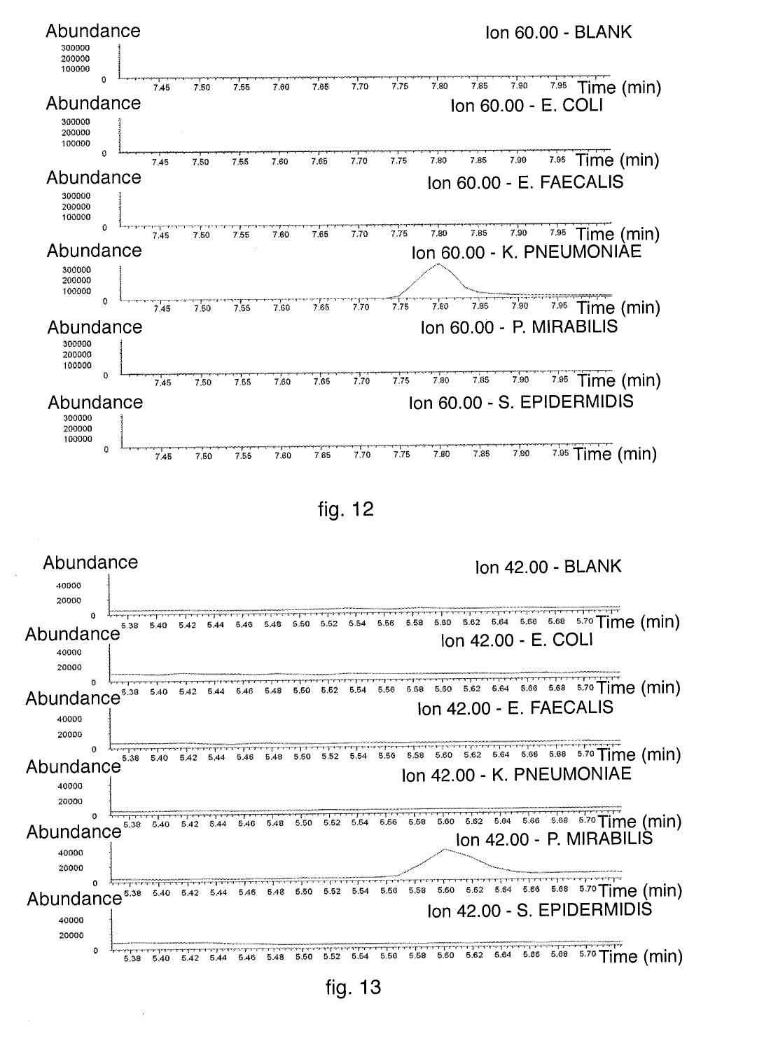

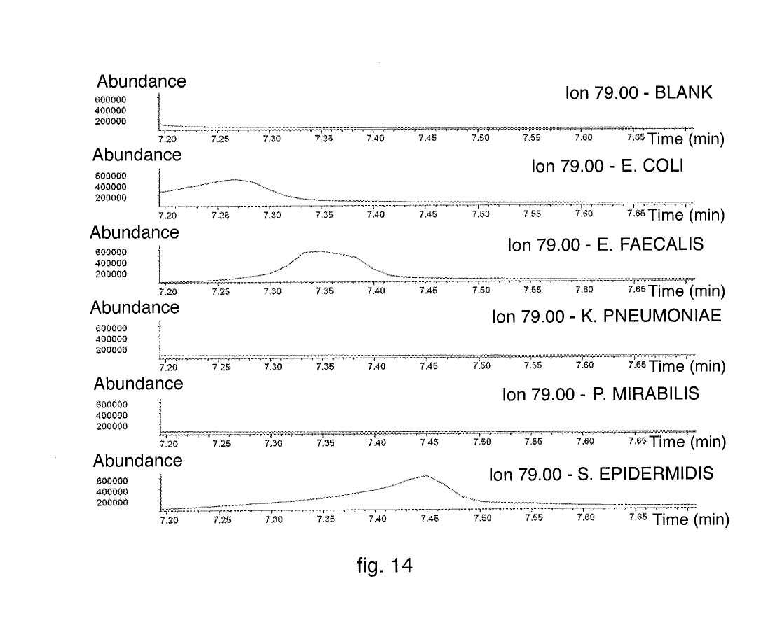

FIGS. 10-14 show examples of plots obtained for native urine, infected respectively with the bacterial strains of E. coli, E. faecalis, K. pneumoniae, P. mirabilis and S. epidermidis.

In these drawings, the RIC diagrams (reconstructed ion chromatograms) of the specific ion classes are compared with those of the non-infected urine.

It can be observed that the class with m/z 117 and a retention time of 24.90 minutes is present only for E. coli, the class with m/z 60 and retention time 8.07 minutes is instead characteristic of E. faecalis. In the same way, the ions with m/z 60 and retention time 7.80 minutes, and with m/z 42 and retention time 5.60 minutes are specific, respectively, for K. pneumoniae and P. mirabilis. The ion with m/z 79 detected for S. epidermidis with a retention time of 7.45 minutes is also present in other strains, but with different retention times, which indicates that it is due to different molecular classes.

Modifications and variants may be made to the present invention, without departing from its field and scope as defined by the following claims.

1. Method to identify bacterial classes in a biological sample, in particular a urine sample, wherein said method provides to sow the biological sample in a culture medium or broth and also provides to carry out the analysis by means of Gas Chromatography-Mass Spectrometry (GC/MS) of the volatile components, such as metabolites and catabolites, of said biological sample in order to identify a graphic plot characteristic of a specific bacterial class, characterized in that the analysis using Gas Chromotography-Mass Spectometry provides to identify the presence of metabolic markers and catabolic markers for each bacterial class, and to construct, by identifying said markers, a specific metabolomic profile and a specific catabolomic profile relating to each bacterial species to be identified, and in that the identification of the specific bacterial class present in the biological sample is obtained by comparing the specific plot obtained by the GC/MS analysis with respect to a plot relating to the specific culture medium without the biological sample.

2. Method as in claim 1, characterized in that the biological samples to be analyzed are inserted into a test tube or vial, closed and sealed, and the volatile components to be then subjected to GC/MS analysis are taken in the volume, or static headspace (SHS), above the biological sample.

3. Method as in claim 1 or 2, characterized in that the biological samples consist of native biological sample.

4. Method as in claim 3, characterized in that said native biological sample is native urine.

5. Method as in claim 1 or 2, characterized in that the biological samples are biological samples enriched with liquid culture broth.

6. Method as in claim 1 or 2, characterized in that the biological samples are biological samples coming from a colony taken from a Petri dish, and diluted in a measuring test tube or vial containing liquid culture broth.

7. Method as in claim 1, characterized in that the identification of the specific bacterial class is obtained by comparing the GC/MS data obtained from infected native urine samples and non-infected native urine samples.

8. Method as in any claim hereinbefore, characterized in that the metabolic peaks (MPP) are determined through the GC/MS analysis, that is, the peaks of the GC/MS plot which identify substances present in the culture medium that the bacterial class has consumed as nourishment for its replication, and the catabolic peaks (CPP), that is, the peaks of the GC/MS plot that identify substances produced by the bacterial class.

9. Method as in claim 8, characterized in that the detection of the metabolic peaks (MPP) of the biological samples and the catabolic peaks (CPP) of the biological samples has been carried out at different degrees of McFarland turbidity, in order to identify the optimal values of bacterial titer for the subsequent GC/MS analysis.

10. Method as in claim 1, characterized in that the samples are taken in the headspace by means of the solid-phase microextraction (SPME) technique.
